(19) United States
(12) Patent Application Publication
(10) Pub. No.: US 2025/0127761 A1
(43) Pub. Date: Apr. 24, 2025

(54) Telmisartan Nanosuspension for Therapy of Respiratory Infections and Methods of Making and Using Same

(71) Applicants: Soligenix, Inc., Princeton, NJ (US); University of Hawaii, Honolulu, HI (US); Johns Hopkins University, Baltimore, MD (US)

(72) Inventors: Oreola Donini, Coquitlam (CA); Axel Lehrer, Honolulu, HI (US); Daiqin Chen, Clarksville, MD (US); Jung Soo Suk, Baltimore, MD (US); Joe DiCostanzo, Honolulu, HI (US)

(21) Appl. No.: 18/837,438

(22) PCT Filed: Feb. 10, 2023
(86) PCT No.: PCT/US2023/012795

§ 371 Date: Aug. 9, 2024

Related U.S. Application Data
(60) Provisional application No. 63/308,874, filed on Feb. 10, 2022.

(51) Int. Cl. A61K 31/4184; A61K 9/00; A61K 47/26

(52) U.S. Cl. CPC A61K 31/4184; A61K 9/0078; A61K 47/26

(57) ABSTRACT

A method of inhibiting viral replication of a virus in an individual comprising administering an effective amount of a drug nanosuspension combined with a surfactant, wherein the drug nanosuspension combined with the surfactant is delivered to the individual's lungs. Preferably, the drug is a nanosuspension delivered to the individual's lungs through inhalation.

### GOVERNMENT SUPPORT STATEMENT

This invention was made with government support under grant no. HL136617, awarded by the National Institutes of Health. The government has certain rights in the invention.

### FIELD OF THE INVENTION

The invention relates to the field of nanosuspension formulations of certain drugs for inhalation therapy of specific viral related conditions.

### BACKGROUND OF THE INVENTION

The outbreak of the coronavirus disease 2019 (COVID-19) pandemic has spurred global efforts to contain the spread of severe acute respiratory syndrome coronavirus 2 (SARS-CoV-2), the causative agent for the disease. Although vaccination is the pre-eminent public health strategy, vaccine hesitancy and the advent of elusive variants have reinforced the need for effective treatment strategies, especially those that prevent the need for hospitalization.

Treatment targeted specifically to the acute respiratory distress syndrome (ARDS) of COVID-19 offers both short-term and long-term utility in the event of escape variants of SARS-CoV-2 and/or other respiratory pandemics. Accumulated evidence suggests that clinically used hypertensive drugs, angiotensin receptor blockers (ARBs), may mitigate deleterious lung pathology, ARDS, in patients with COVID-19, presumably via modulation of the renin-angiotensin system (RAS) perturbed by the disease [1-4].

Specifically, ARBs can shift the pro-inflammatory angiotensin II (ANG II)-dominant pathological state of ARDS towards an anti-inflammatory angiotensin converting enzyme 2 (ACE2)-dominant state [5]. Further, potential inhibitory roles of ARBs on replication of SAR-CoV-2 have been recently suggested and experimentally validated in vitro [6-8]. Thus, ARBs could exert meaningful therapeutic intervention in COVID-19 lungs in a multi-modal manner.

However, universal use of oral ARB formulations poses safety concerns due to the established systemic adverse effects, particularly for those with normal blood pressure or hypotension [9-12]. Additionally, unlike the original use for systemic pressure-reducing effects, a very high oral dose is likely needed to achieve desirable therapeutic concentrations in the lung tissue to yield meaningful clinical outcomes.

There remains a need in the art to have an inhalable formulation of a clinically used angiotensin receptor blocker which can be administered directly into the lung, in a safe and effective manner.

### SUMMARY OF THE INVENTION

The present invention provides for a method of inhibiting viral replication of a virus in an individual comprising administering an effective amount of a drug nanosuspension combined with a surfactant, wherein the drug nanosuspension combined with the surfactant is delivered to the individual's lungs. Preferably, the surfactant is Polysorbate 80. More preferably, the drug is telmisartan or a pharmaceutically active salt thereof. Preferably, the drug nanosuspension is delivered to the individual's lungs through inhalation.

In another embodiment, the present invention provides for a drug nanosuspension comprising at least one drug and at least one surfactant, wherein the at least one drug and at least one surfactant are combined in powder form prior to reconstitution in an aqueous solution for inhalation administration to an individual in need thereof.

### BRIEF DESCRIPTION OF THE DRAWINGS

The following figures provide illustrative examples of the present invention and are incorporated by reference within this disclosure.

FIG. 1A-1F show physicochemical properties and in vitro anti-virus activity of INH-TEL. (A) Hydrodynamic diameters of freshly prepared (black) and lyophilized-rehydrated (red) INH-TEL measured by DLS. (B) Representative transmission electron micrographs of freshly prepared (left) and lyophilized-rehydrated (right) INH-TEL. Scale bar=100 nm. (C) Colloidal stability of INH-TEL in a physiological lung environment determined by the changes of particle hydrodynamic diameters in mouse BALF at 37° C. over time (n=3 independent experiments). (D) Cumulative in vitro release of the drug payloads (i.e., telmisartan) from INH-TEL in DPBS supplemented with 0.05% Tween 80 over time (n=3 independent experiments). (E) In vitro drug activity (i.e., inhibition of intracellular calcium spike induced by ANG II) of INH-TEL in comparison to FD-TEL. (F) In vitro inhibitory effect on SARS-CoV-2 replication (red) and cytotoxicity (black) of INH-TEL in Calu-3 (n=3 independent experiments). *p<0.05 (one-way ANOVA).

FIG. 2A-2D show pharmacokinetics of INH-TEL following intratracheal administration into the lungs of wild-type C57BL/6 mice and cynomolgus macaques. (A) Lung and plasma concentrations of telmisartan 1 and 12 hour(s) after a single intratracheal administration of INH-TEL at a telmisartan dose of 0.1 mg/kg into the lungs of wild-type C57BL/6 mice (n=5 mice per group). (B-D) Two macaques, including CM1 (3.08 kg) and CM2 (2.86 kg), received a single intratracheal (IT) administration of INH-TEL at a fixed telmisartan dose of 2.5 mg (0.81-0.87 mg/kg, calculated based on the body weights) and one macaque (CM3; 3.08 kg) received daily oral gavage (OG) administration of FD-TEL at a telmisartan dose of 1 mg/kg for 7 days. (B) Telmisartan content in the lung tissues from CM1 and CM2 harvested at 0.5- and 8-hour post-administration of INH-TEL into the lung, respectively, and from CM3 harvested 2 hours after the last (i.e., 7th) daily oral administration of FD-TEL. (C) Plasma pharmacokinetics of telmisartan in CM1 and CM2 received a single intratracheal administration of INH-TEL and CM3 received the 6th daily oral administration of FD-TEL. Plasma pharmacokinetics were monitored until CM1 and CM2 were euthanized to harvest lung tissues and up to 12 hours for CM3. (D) Relative telmisartan content in the lung tissue versus the plasma harvested from CM1 and CM2 at 0.5- and 8-hour post-administration of INH-TEL into the lung. *p<0.05, ***p<0.005 and ****p<0.001 (one-way ANOVA).

FIG. 3 shows histopathological analysis of lung tissues from macaques received either intratracheal INH-TEL or oral FD-TEL. Lung tissues were harvested at different time points after a single intratracheal administration of INH-TEL at a fixed telmisartan dose of 2.5 mg (0.81-0.87 mg/kg) or after the 7th oral gavage administration of FD-TEL at a telmisartan dose of 1 mg/kg.

### DETAILED DESCRIPTION OF THE INVENTION

The present invention provides for a formulation of an inhalable telmisartan formulation (INH-TEL) composed of TEL drug nanosuspension core stably coated by Polysorbate 80.

### Examples

The following examples illustrate the various embodiments of the present invention and are not meant to be limiting in scope based on such examples.

### A. Physiochemical Characterization of INH-TEL

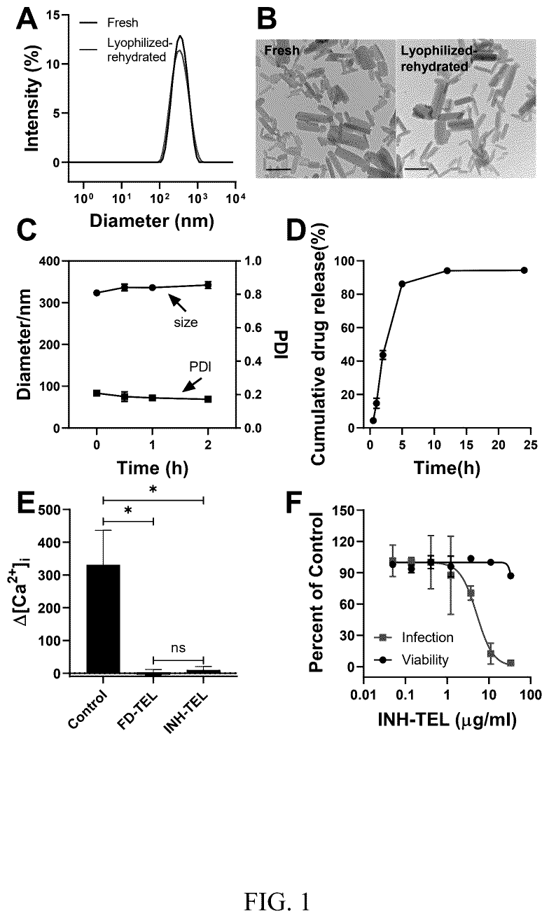

A physicochemical characterization was first conducted to measure the hydrodynamic diameters and ζ-potentials of freshly prepared INH-TEL to be 290±30 (polydispersity index or PDI=0.20±0.04) nm and −2.9±0.9 mV, respectively (FIG. 1A and Table 1). The ratio between telmisartan and Polysorbate 80 of the final product was roughly 4:1 (Table 1).

TABLE 1
Physicochemical properties of freshly prepared
and lyophilized-rehydrated INH-TEL.

| | Hydrodynamic | | ζ-potential | Drug loading |
|---|---|---|---|---|
| INH-TEL | diameter (nm) | PDI | (mV) | density (%) |
| Fresh | 290 ± 30 | 0.20 ± 0.04 | −2.9 ± 0.9 | 82 ± 3 |
| Lyophilized- | 359 ± 21 | 0.35 ± 0.12 | −8.1 ± 2.2 | — |
| rehydrated | | | | |

It was then identified that lyoprotectant-free lyophilization and subsequent reconstitution (i.e., rehydration) did not yield particle aggregates and resulted in only moderate changes in hydrodynamic diameters (359±21 nm; PDI=0.35±0.12) and ζ-potentials (−8.1±2.2 mV) (FIG. 1A and Table 1). Likewise, transmission electron microscopy revealed that both freshly prepared and lyophilized-rehydrated INH-TEL possessed rod-shaped morphology with similar geometric sizes (FIG. 1B).

The findings here underscore that INH-TEL could be stored long-term in a powder form prior to reconstitution in an aqueous vehicle solution for inhaled administration. INH-TEL also demonstrated excellent colloidal stability in a physiologically relevant lung environment, mouse bronchoalveolar lavage fluid, at least up to 2 hours, as evidenced by negligible changes in hydrodynamic diameters and PDI (FIG. 1C).

In parallel, an in vitro drug release study was conducted using Dulbecco's phosphate-buffered saline (DPBS) supplemented with 0.05% Polysorbate 80 as an artificial sink condition, where nearly 90% of telmisartan was released within the first 5 hours (FIG. 1D). The rapid drug release may be beneficial for managing acute pathological conditions that require prompt drug action, such as ARDS triggered by respiratory pathogens.

Testing was then initiated to determine whether the drug release from INH-TEL preserved its inherent drug activity by assessing the ability to prevent ANG II-mediated elevation of intracellular calcium ion concentration ([Ca2+]i) in lung smooth muscle cells. ANG II binding to its cell surface receptor (i.e., ANG II type 1 receptor) activates the voltage-gated Ca2+ channels to elevate [Ca2+]i, which is effectively inhibited by ARBs [13, 14]. It was discovered that ANG II-mediated transient [Ca2+]i spike was equally and entirely abrogated when cells were treated with dose-matched FD-TEL or INH-TEL (FIG. 1E), suggesting that the drug activity of INH-TEL was fully retained.

To test the hypothesis that INH-TEL might provide anti-viral efficacy, we then assessed the ability of INH-TEL to deter SARS-CoV-2 replication in vitro. It was found that the viral replication was inhibited by INH-TEL in a dose-dependent manner (up to the telmisartan concentration of 33.3 g/ml) in Calu-3 cells without incurring significant cytotoxicity (FIG. 1F). Of note, Calu-3 has been confirmed for expression of the cell surface portal for SARS-CoV-2 (i.e., ACE2) and susceptibility to the viral infection accordingly [15]. This observation agrees with recent reports demonstrating inhibitory effects of various ARBs, including telmisartan, against SARS-CoV-2 replication in Vero-E6 or Caco-2 cells [6-8]. In relevance to these finding, it has been shown that intracellular calcium is essential for viral assembly and budding of SARS-CoV [16], the virus responsible for outbreak of severe acute respiratory syndrome in 2003, and that ARB reduces viral spread by preventing release of several enveloped viruses from infected cells [17-19]. More recently, potential role of ARBs on blocking main protease of SARS-CoV-2 essential for viral replication and transcription has been suggested [7], but further investigation is warranted to fully unravel the mechanism(s) of inhibition. It is noteworthy that, while ARBs were initially speculated to upregulate ACE2 to promote viral infection and disease severity, recent independent studies refuted such a hypothesis [20].

B. Telmisartan Concentrations in the Lung Measured after Direct Administration of INH-TEL The next determination was an evaluation of the hypothesis that direct administration of INH-TEL into the lung would provide high telmisartan concentrations in the lung. To test this, INH-TEL was intratracheally administered into the lungs of C57BL/6 mice at a telmisartan dose of 0.1 mg/kg and compared the drug content in the lung and the plasma at different time points.

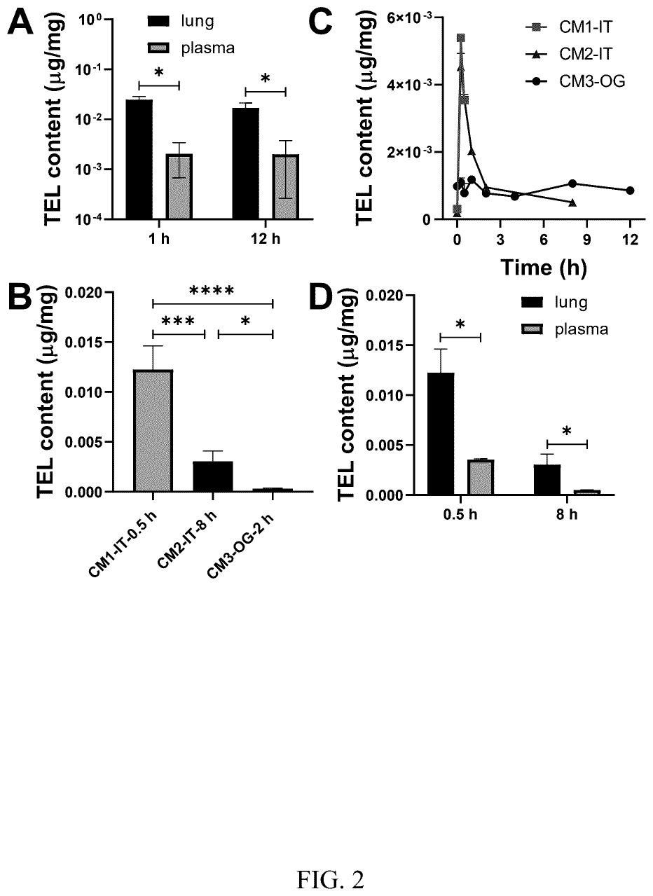

It was discovered that telmisartan content was about an order of magnitude greater in the lung compared to the plasma at 1- and 12-hour post-administration (FIG. 2A).

To complement this mouse study, a pharmacokinetic study was conducted using non-human primates (i.e., cynomolgus macaques) in which locally administered INH-TEL was compared to oral FD-TEL. Specifically, two macaques were intratracheally treated with INH-TEL at a telmisartan dose of 2.5 mg per animal (0.81-0.87 mg/kg) and lung tissues were harvested at 0.5 or 8-hour post-administration for the assessment of drug content in the lung. As a clinically relevant control, one macaque received daily oral FD-TEL for 7 days at a telmisartan dose of 1 mg/kg and lung tissue was harvested 2 hours after the final dose. Macaques received intratracheal INH-TEL, regardless of the time of lung harvest, exhibited at least 10-fold greater drug content in the lung compared to the orally treated macaque (FIG. 2B). In parallel, plasma pharmacokinetics of these animals was monitored. It was determined that plasma drug content of the animals receiving intratracheal INH-TEL was transiently elevated but quickly reduced to the level on par with or lower than the steady-state plasma drug content observed with the animals under the daily oral FD-TEL regimen (FIG. 2C). In agreement with the mouse study (FIG. 2A), telmisartan content was markedly and significantly greater in the lung compared to the plasma at both 0.5- and 8-hour post-administration (FIG. 2D).

### C. Tolerability of Intratracheally Administered INH-TEL in Non-Human Primates

Tolerability of intratracheally administered INH-TEL in non-human primates was also examined. Specifically, randomly selected parts of lung tissues harvested from three macaques at the respective times of pulmonary drug content analysis (FIG. 2C) were subjected to paraffin section and hematoxylin & eosin staining. The lung slides were then scored in a blinded manner for edema, composite inflammation, increased bronchus-associated lymphoid tissue (BALT), reactive epithelial changes, alveolar collapse, and interstitial fibrosis by a board-certified pathologist (KT).

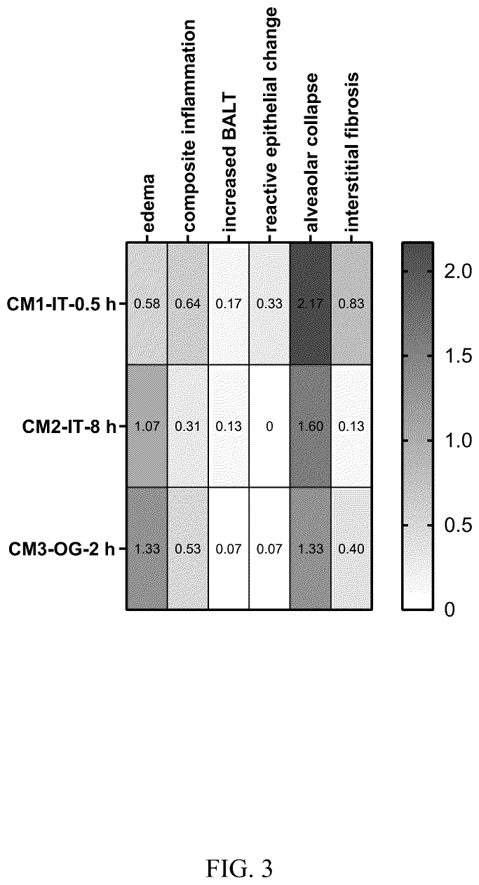

The evaluation revealed no significant histopathologic differences between intratracheally administered INH-TEL and oral FD-TEL with acceptable tolerability in the lung tissues (FIG. 3). It was noted that mild alveolar collapse observed shortly after the intratracheal administration of INH-TEL was quickly resolved (FIG. 3). Blood biochemistry analysis was also conducted at the times of lung harvests and respective baselines (i.e., prior to the administration). It was determined that most of the biochemical readouts were comparable before and after the treatments (Table 1), indicating that the formulation did not exert significant systemic toxicity. Although a significant increase of creatine kinase was observed (Table 2), it is unlikely due to the formulation, given that the perturbation was present regardless of treatment type and is not readily expected from damaged lung tissues. It was suspected that the elevation resulted from hemolysis [121] and muscle stress/damage [122] caused by venous puncture and physical restraint, respectively, which are associated with the treatment procedure and/or blood drawing.

TABLE 2
Blood biochemistry panel of macaques received
either intratracheal INH-TEL or oral FD-TEL.

| | Macaque ID | | | | | | | |
|---|---|---|---|---|---|---|---|---|
| | CM1 | | CM2 | | CM3 | | | |
| | Administration | | | | | | | |
| | IT | | IT | | OG | | | |
| | Treatment | | | | | | | |
| | INH-TEL (2.5 mg) | | INH-TEL (2.5 mg) | | FD-TEL (1 mg/kg) | | | |
| | Time | | | | | | | |
| | | | | | | D 6 | D 7 | |
| | B.L.^{†} | 0.5 h^{§} | B.L.^{†} | 8 h^{§} | B.L.^{†} | B.L.^{‡} | B.L.^{‡} | 2 h^{§} |
| ALP (U/L) | 137 | 128 | 135 | 147 | 164 | 150 | 158 | 155 |
| ALT (U/L) | 64 | 61 | 15 | 51 | 86 | 131 | 177 | 176 |
| AST (U/L) | 44 | 47 | 23 | 235 | 85 | 86 | 163 | 222 |
| Albumin (g/dL) | 3.8 | 3.5 | 3.7 | 3.8 | 3.5 | 3.4 | 3.6 | 3.5 |
| Total Protein (g/dL) | 6.8 | 6.3 | 6.7 | 7 | 6.9 | 6.2 | 6.4 | 6.2 |
| Globulin (g/dL) | 3 | 2.8 | 3 | 3.2 | 3.4 | 2.8 | 2.8 | 2.7 |
| Total Bilirubin | 0.2 | 0.2 | 0.1 | 0.3 | 0.1 | 0.2 | 0.2 | 0.2 |
| (mg/dL) | | | | | | | | |
| Bilirubin-Conjugated | 0 | 0 | 0 | 0.1 | 0 | 0.1 | 0.1 | 0 |
| (mg/dL) | | | | | | | | |
| BUN (mg/dL) | 31 | 27 | 19 | 20 | 26 | 22 | 26 | 27 |
| Creatinine (mg/dL) | 0.7 | 0.6 | 0.6 | 0.5 | 0.7 | 0.6 | 0.8 | 0.8 |
| Cholesterol (mg/dL) | 133 | 127 | 125 | 137 | 125 | 106 | 103 | 104 |
| Glucose (mg/dL) | 79 | 100 | 87 | 100 | 97 | 77 | 85 | 59 |
| Calcium (mg/dL) | 9 | 8.8 | 9.1 | 8.7 | 9.6 | 8.9 | 9.7 | 9.3 |
| Phosphorus (mg/dL) | 3.9 | 3.7 | 3.7 | 5.6 | 5 | 3.1 | 3 | 3.2 |
| Chloride (mmol/L) | 104 | 106 | 109 | 113 | 105 | 105 | 103 | 108 |
| Potassium (mmol/L) | 3.3 | 3.8 | 3.5 | 3.7 | 3 | 3.8 | 4.1 | 3.4 |
| Sodium (mmol/L) | 146 | 146 | 148 | 149 | 146 | 147 | 145 | 148 |
| ALB/GLOB ratio | 1.3 | 1.3 | 1.2 | 1.2 | 1 | 1.2 | 1.3 | 1.3 |
| BUN/Creatinine Ratio | 44.3 | 45 | 31.7 | 40 | 37.1 | 36.7 | 32.5 | 33.8 |
| Bilirubin- | 0.2 | 0.2 | 0.1 | 0.2 | 0.1 | 0.1 | 0.1 | 0.2 |
| Unconjugated | | | | | | | | |
| (mg/dL) | | | | | | | | |
| NA/K Ratio | 44 | 38 | 42 | 40 | 49 | 39 | 35 | 44 |
| Hemolysis Index | N | + | N | +++ | N | + | N | N |
| Lipemia Index | N | N | N | N | N | N | N | N |

^{†}Baseline levels prior to the intratracheal administration of INH-TEL or the first oral administration of FD-TEL.
^{‡}Baseline levels prior to 6^{th} or 7^{th} daily oral dose of FD-TEL.
^{§}Levels at different times post-administration of intratracheal INH-TEL or of the last (i.e., 7^{th}) oral administration of FD-TEL.

In summary, a surface-stabilized nanosuspension formulation of telmisartan capable of long-term storage and shipping in a powder form was developed and experimentally confirmed with respect to its physiological stability, unperturbed drug activity and inhibitory potential against SARS-CoV-2 infection. Further, the formulation of the present invention demonstrates excellent lung pharmacokinetics and acceptable local and systemic tolerability as revealed by the above-described non-human primate studies. This suggests an expectation of success of this nanosuspension formulation in humans based on the above identified primate study results.

As used in this specification and in the appended claims, the singular forms include the plural forms. For example, the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

### REFERENCES

1. Zeinalian, M., et al., A potential protective role of losartan against coronavirus-induced lung damage. Infect Control Hosp Epidemiol, 2020. 41(6): p. 752-753.
2. Rothlin, R. P., et al., Telmisartan as tentative angiotensin receptor blocker therapeutic for COVID-19. Drug Dev Res, 2020. 81(7): p. 768-770.
3. Duarte, M., et al., Telmisartan for treatment of Covid-19 patients: An open multicenter randomized clinical trial. EClinicalMedicine, 2021. 37: p. 100962.
4. Rothlin, R. P., et al., Angiotensin Receptor Blockers for COVID-19: Pathophysiological and Pharmacological Considerations About Ongoing and Future Prospective Clinical Trials. Front Pharmacol, 2021. 12: p. 603736.
5. Zaheer, J., H. Kim, and J. S. Kim, Correlation of ACE2 with RAS components after Losartan treatment in light of COVID-19. Sci Rep, 2021. 11(1): p. 24397.
6. Nejat, R., et al., Losartan Inhibits SARS-CoV-2 Replication in Vitro. J Pharm Pharm Sci, 2021. 24: p. 390-399.
7. Alnajjar, R., et al., Molecular docking, molecular dynamics, and in vitro studies reveal the potential of angiotensin II receptor blockers to inhibit the COVID-19 main protease.

Heliyon, 2020. 6(12): p. e05641.
8. Reus, P., et al., Characterization of ACE Inhibitors and ATIR Antagonists with Regard to Their Effect on ACE2 Expression and Infection with SARS-CoV-2 Using a Caco-2 Cell Model. Life (Basel), 2021. 11(8).
9. Yusuf, S., et al., Irbesartan inpatients with atrial fibrillation. N Engl J Med, 2011. 364(10): p. 928-38.
10. Pitt, B., Adverse events in HEAAL: when to hold and when to fold. Eur J Heart Fail, 2012. 14(12): p. 1319-21.
11. Mann, J. F., et al., Effect of telmisartan on renal outcomes: a randomized trial. Ann Intern Med, 2009. 151(1): p. 1-10.
12. Kiernan, M. S., et al., Predicting adverse events during angiotensin receptor blocker treatment in heart failure: results from the HEAAL trial. Eur J Heart Fail, 2012. 14(12): p. 1401-9.
13. Shimoda, L. A., et al., Inhibition of hypoxia-induced calcium responses in pulmonary arterial smooth muscle by acetazolamide is independent of carbonic anhydrase inhibition. American Journal of Physiology-Lung Cellular and Molecular Physiology, 2007. 292(4): p. 1002-1012.
14. Abadir, P. M., et al., Angiotensin II Type-2 receptors modulate inflammation through signal transducer and activator of transcription proteins 3 phosphorylation and TNFα production. Journal of Interferon and Cytokine Research, 2011. 31(6): p. 471-474.
15. Saccon, E., et al., Cell-type-resolved quantitative proteomics map of interferon response against SARS-CoV-2. iScience, 2021. 24(5): p. 102420.
16. Lu, W., K. Xu, and S. B., SARS Accessory Proteins ORF3a and 9b and Their Functional Analysis. Molecular Biology of the SARS-Coronavirus. 2009.
17. Choi, J. A., et al., The effects of losartan on cytomegalovirus infection in human trabecular meshwork cells. PLoS One, 2019. 14(6): p. e0218471.
18. Gardner, P. L., G. N. Mbuy, and M. T. Knabb, Effects of the angiotensin II receptor antagonist losartan on herpes simplex virus-type 2 infection of cultured vero and cardiac neonatal myocytes. Life Sci, 1994. 55(4): p. 283-9.
19. Bai, D., et al., Porcine deltacoronavirus (PDCoV) modulates calcium influx to favor viral replication. Virology, 2020. 539: p. 38-48.
20. Jackson, C. B., et al., Mechanisms of SARS-CoV-2 entry into cells. Nat Rev Mol Cell Biol, 2022. 23(1): p. 3-20.
21. Greenson, J. K., S. J. Farber, and S. B. Dubin, The effect of hemolysis on creatine kinase determination. Arch Pathol Lab Med, 1989. 113(2): p. 184-5.
22. Bennett, J. S., et al., Effects of ketamine hydrochloride on serum biochemical and hematologic variables in rhesus monkeys (Macaca mulatta). Vet Clin Pathol, 1992. 21(1): p. 15-18.

We claim:

1. A method of inhibiting viral replication of a virus in an individual comprising administering an effective amount of a drug nanosuspension combined with a surfactant, wherein the drug nanosuspension combined with the surfactant is delivered to the individual's lungs.

2. The method of claim 1, wherein the surfactant is Polysorbate 80.

3. The method of claim 1, wherein the drug is telmisartan or a pharmaceutically active salt thereof.

4. The method of claim 1, wherein the drug nanosuspension is delivered to the individual's lungs through inhalation.

5. A drug nanosuspension comprising at least one drug and at least one surfactant, wherein the at least one drug and at least one surfactant are combined in powder form prior to reconstitution in an aqueous solution for inhalation administration to an individual in need thereof.

6. A method of mitigating lung damage in an individual at risk of acute respiratory distress syndrome comprising administering an effective amount of a drug nanosuspension combined with a surfactant, wherein the drug nanosuspension combined with the surfactant is delivered to the individual's lungs.

7. The method of claim 6, wherein the surfactant is Polysorbate 80.

8. The method of claim 6, wherein the drug is telmisartan or a pharmaceutically active salt thereof.

9. The method of claim 6, wherein the drug nanosuspension is delivered to the individual's lungs through inhalation.

10. A drug nanosuspension comprising at least one drug and at least one surfactant, wherein the at least one drug and at least one surfactant are combined in powder form prior to reconstitution in an aqueous solution for inhalation administration to an individual in need thereof.
